(19) United States
(12) Patent Application Publication
(10) Pub. No.: US 2015/0250888 A1
(43) Pub. Date: Sep. 10, 2015

(54) PROCESS FOR PREPARING AQUEOUS DISPERSIONS

(71) Applicants: Shraddha Sanjeev Joshi, (US); Ashish Sharadchandra GUHA, (US); Vinay JAIN, Indore (M.P) (IN)

(72) Inventors: Shraddha Sanjeev Joshi, Navi Mumbai (IN); Ashish Sharadchandra Guha, Mumbai (IN); Vinay Jain, Indore (M.P) (IN)

(73) Assignee: Evonik Industries AG, Essen (DE)

(21) Appl. No.: 14/430,592

(22) PCT Filed: Nov. 27, 2012
(86) PCT No.: PCT/EP2012/073705

§ 371 Date: Mar. 24, 2015

(30) Foreign Application Priority Data
Sep. 28, 2012 (IN) 4063/CHE/2012

(51) Int. Cl. A61K 47/32; A61K 9/10

(52) U.S. Cl. CPC A61K 47/32; A61K 9/10

(57) ABSTRACT

The invention discloses a process for preparing aqueous dispersions comprising the components (a) and (b) and optionally (c) or (d) or both, (a) a copolymer consisting of free-radical-polymerized C1- to C4-esters of acrylic or methacrylic acid and alkyl(meth)acrylate monomers with tertiary ammonium groups in the alkyl side groups, (b) 5 to 25% by weight, based on (a), of a C₁₂₋ to C₁₈-monocarboxylic acid or a C₁₂₋ to C₁₈-alcohol, (c) 0 to 10% by weight based on (a) of a dicarboxylic acid having 3 to 10 carbon atoms, (d) 0 to 20% by weight, based on (a), of an emulsifier having an HLB of at least 14 by dispersing the components in water where the water has a temperature of more than 40 and up to 85° C., where the solid content of the dispersion is from 5 to 40% by weight and where a clear colloidal dispersion is obtained in less than 30 min.

### FIELD OF THE INVENTION

The inventions describes a process for preparing aqueous dispersions comprising an amino(meth)acrylate copolymer.

### TECHNICAL BACKGROUND

U.S. Pat. No. 4,705,695 describes a method for coating pharmaceutical formulations comprising a dissolved polymer prepared from acrylic and/or methacrylic monomers having a side group containing a tertiary amine substituent, where the amine nitrogen atom is converted to the ammonium salt form and then drying said coated film. A copolymer with the monomer composition of EUDRAGIT® E is suspended in powder form in water. The suspension was stirred vigorously for about 2 hours until a slightly turbid, yellowish solution resulted.

U.S. Pat. No. 4,737,357 describes an aqueous coating dispersion of a water-swellable but water-insoluble polymer formed between a quaternary ammonium monomer and a nonionic monomer. The polymer is dispersed in the presence of a plasticizer as a powder in water under gentle stirring and elevated temperatures ranging from 50 to 100° C. The preparation times take several hours.

EP 109893581 describes a coating and excipient agent for oral or dermal delivery. Dispersions of EUDRAGIT® E100 are achieved by converting the polymer granulate into a powder. The polymer powder is mixed at room temperature in water together with a detergent, polysorbat 80, and a plasticizer, triethylcitrate, under gentle stirring for about 90 min. Subsequently glycerol monostearate is added and the mixture is homogenized under high speed stirring with an Ultra-Turrax® equipment for 10 min at 3.500 rpm. In another example antifoam agents are added in the high speed stirring phase.

WO 02/67906 describes a process for the production of a coating and binding agent for oral or dermal pharmaceutical forms consisting essentially of

(a) a copolymer consisting of free-radical-polymerized C1- to C4-esters of acrylic or methacrylic acid and further (meth)acrylate monomers which have functional tertiary ammonium groups, the copolymer being present in powder form having an average particle size of 1-40 μm (e.g. EUDRAGIT® E PO)
(b) 3 to 15% by weight, based on (a), of an emulsifier having an HLB of at least 14
(c) 5 to 50% by weight, based on (a), of a C₁₂- to C₁₈-monocarboxylic acid or a C₁₂- to C₁₈-hydroxyl compound,
the components (a), (b) and (c) being blended or mixed with one another with or without addition of water and optionally with addition of a pharmaceutical active compound and further customary additives and the coating and binding agent being produced from the mixture by melting, casting, spreading, spraying or granulating. The dispersing times of mixtures of EUDRAGIT® E PO with sodium laurylsulfate and stearic acid in the examples is ranging from 2 to 5 hours. Stirring is performed with a bladed stirrer at about 400 rpm.

WO 2011/012161A1 describes a powdery or granulated composition comprising at least by 30% by weight of a mixture of the components (a), (b) and (c) with

(a) a copolymer composed of polymerized units of C₁- to C₄-alkyl esters of acrylic or methacrylic acid and of alkyl(meth)acrylate monomers with a tertiary amino group in the alkyl radical and
(b) 0.5 to 10% by weight based on (a) of a dicarboxylic acid having 3 to 10 carbon atoms and
(c) 5 to 20% by weight based on (a) of a fatty monocarboxylic acid having 8 to 18 carbon atoms.

The inventive composition is intended to be used as a rapidly in water dissolving powder or granulate. The dispersed aqueous compositions show a low viscosity and can therefore be processed directly as coating and binding agents for pharmaceutically, nutraceutically or cosmetically purposes. The components (a), (b) and (c) are added to the aqueous dispersing or solution agent, preferably purified water, as powder mixtures, granules or single one after another while gentle stirring with a conventional stirrer at room temperature. Advantageously, according to this invention, the need of a high shear mixer or specific disperser will not be necessary. Additionally, the heating of the suspension will be not necessary. After stirring of less than 3 hours, dispersions or solutions are formed being able to be sprayed in coating or granulation processes and/or to form films after drying. The preparation times for the dispersions in the examples range from 0.2 to 2.8 hours (example 9/24). Stirring is performed with a magnetic stirrer or a simple agitator providing low shear forces. A combination of EUDRAGIT® E PO with 3% by weight tartaric acid and 15% by weight stearic acid (example 19) has a preparation time of 1.8 hours.

WO 2011/012335A1 describes a powdery or granulated composition comprising at least by 30% by weight of a mixture of

(a) a copolymer composed of polymerized units of C₁- to C₄-alkyl esters of acrylic or methacrylic acid and of alkyl(meth)acrylate monomers with a tertiary amino group in the alkyl radical and
(b) 5 to 15% by weight based on (a) of a salt of a fatty monocarboxylic acid having 10 to 18 carbon atoms, and
(c) 10 to 20% by weight based on (a) of fatty monocarboxylic acid having 8 to 18 carbon atoms and/or a fatty alcohol having 8 to 18 carbon atoms.

The inventive composition is intended to be used as a rapidly in water dissolving powder or granulate. The dispersed aqueous compositions show a low viscosity and can therefore be processed directly as coating and binding agents for pharmaceutically, nutraceutically or cosmetically purposes. The preparation times for the dispersions in the examples range from 0.5 to 2.5 hours.

The EUDRAGIT® Application Guidelines, Edition 12.0, Chapter 7.2, describe the formulation basis for taste masked formulations. A formulation containing EUDRAGIT® E PO, 10% sodium lauryl sulfate (SLS), 15% stearic acid and 50% talc is described. The formulation may be prepared as a spray solution. First, the water is put into a vessel. SLS and stearic acid are added quickly, then EUDRAGIT® E PO is added at once by complete coverage of the liquid surface. Following this order enhances the polymer dissolution and helps to avoid foam formation. Continue to stir at medium speed, avoiding a vortex, in order to keep foam formation low. After EUDRAGIT® E PO has been added, the vessel can be covered. During continuous stirring, the polymer particles dissolve resulting in a slightly yellowish, ultra-fine dispersion. The dissolution process lasts around 1-1.5 hours. Using heated water (up to 40° C.) the preparation time can be significantly decreased. Talc and pigments are then added and dispersed with continuous stirring. In order to speed up the preparation, co-milling of SLS and stearic acid may be considered as a pre-processing step. Separate homogenization of talc and pigments may be considered.

### Problem and Solution

Copolymers consisting of free-radical-polymerized C1- to C4-esters of acrylic or methacrylic acid and alkyl(meth)acrylate monomers with tertiary ammonium groups in the alkyl side groups, the copolymer being present in powder form having an average particle size of 1-40 μm, are well known in the pharmaceutical and the neutraceutical industry for instance under the trade name EUDRAGIT® E PO.

EP 109893581 describes a coating and excipient agent for oral or dermal delivery. Dispersions of EUDRAGIT® E100 are achieved by converting the polymer granulate into a powder. The polymer powder is mixed at room temperature in water together with a detergent, polysorbat 80, and a plasticizer, triethylcitrate, under gentle stirring for about 90 min. Subsequently glycerol monostearate is added and the mixture is homogenized under high speed stirring with an ultra turrax equipment for another 10 min at 3.500 rpm. In another example antifoam agents are added in the high speed stirring phase.

WO 02/67906 describes a process for the production of a coating and binding agent for oral or dermal pharmaceutical forms based on the polymer type EUDRAGIT® E. The dispersing times at room temperature of mixtures of EUDRAGIT® E PO (powdered form) with sodium laurylsulfate and stearic acid in the examples is ranging from 2 to 5 hours. The dispersion preparation time for of EUDRAGIT® E PO with 10% sodium laurylsulfate and 15% stearic acid is for instance indicated to be about 6 hours (example 9). A composition of EUDRAGIT® E PO with 7% sodium laurylsulfate and 30% stearic acid (example 7) at a combination of room temperature to 73° C. is mentioned to take about 4 hours. Stirring is performed in all cases with a bladed stirrer at about 400 rpm.

A composition of EUDRAGIT® E PO with 10% sodium laurylsulfate and 15% stearic acid is commonly used for the coating of pharmaceutical compositions or for the binding of active ingredients in matrix systems. It's a permanent effort to reduce the preparation times for making aqueous dispersions as much as possible. The EUDRAGIT® Application Guidelines, Edition 12.0, Chapter 7.2 describe for this kind of formulation dispersion times of 1 to 1.5 hours. By using heated water (up to 40° C.) the preparation time can be significantly decreased.

The application of higher water temperatures is known from WO 02/67906 to be not necessary and to be of low efficiency. The application of high-shear mixers is usually avoided because of problem that large amounts of foam are generated by this way. EP 1098935B1 recommends gentle stirring and whenever high shear mixing is used antifoam agents are recommended to reduce the foam. However its is a general approach i in the pharmaceutical and the nutraceutical industry to avoid the addition of any kind of further agents whenever possible and to reduce to number of ingredients as much as possible.

It was an object of the present invention to provide a process for preparing an aqueous dispersion based on a copolymer consisting of free-radical-polymerized C1- to C4-esters of acrylic or methacrylic acid and alkyl(meth)acrylate monomers with tertiary ammonium groups in the alkyl side groups, the copolymer being present in powder form having an average particle size of 1-40 μm, where the preparation times are further reduced and at the same time the formation of foam is avoided or at least reduced and where the addition of further processing excipients like antifoam agents, are avoided.

The problem was solved according to the features of claim 1.

### DETAILED DESCRIPTION OF THE INVENTION

### Definitions

### High-Shear Mixer

The terms high-shear mixer, high-shear mixing or high-shear homogenisation are well known to a skilled person in the field of pharmacy or galencis.

A definition for a term high-shear mixer may be found for instance in Wikipedia (http:/en.wikipedia.org/wiki/Highshear mixer):

A high-shear mixer disperses, or transports, one phase or ingredient (liquid, solid, gas) into a main continuous phase (liquid), with which it would normally be immiscible. A rotor or impellor, together with a stationary component known as a stator, or an array of rotors and stators, is used either in a tank containing the solution to be mixed, or in a pipe through which the solution passes, to create shear.

A highshear mixer can be used to create emulsions, suspensions, lyosols (gas dispersed in liquid), and granular products. It is used in the adhesives, chemical, cosmetic, food, pharmaceutical, and plastics industries for emulsification, homogenization, particle size reduction, and dispersion.

A well known and broadly used type of a high-shear mixer is for instance the ULTRA-TURRAX® type. The stirring speed may be at least 1000 rpm, preferably 2000-4000 rpm.

### Clear Colloidal Dispersion

A clear colloidal dispersion is obtained when drops of the dispersing aqueous mixture, which may be taken for instance every 5 minutes and are observed under a polarization microscope with a magnification of 100-fold with the support of a phase filter, no or almost no particles (at least less than ten particles in the view field) are observed in the fluid of such a drop under the microscope. This may be taken as the end point of the dispersion process. At this time point a clear colloidal dispersion is obtained. The accuracy of this determination method is in most cases sufficient to differ the preparation times of the different dispersion preparations apart from each other.

The inventive process may be characterized in that a clear colloidal dispersion is obtained in less than 30 min, preferably in less than 25 min, in less than 20 min or in less than 15 min.

The preparation time for making a clear colloidal dispersion is starting from adding the dry powdery or granulate components one by one or together as a mixture into the preheated water under stirring or shear application.

### Mean Particle Size

The mean particle size of the powders, for instance that of component (a), can be determined as follows: By air-jet screening for simple separation of the ground product into a few fractions. In the present measurement range, this method is somewhat less accurate than the alternatives. At least 70%, preferably 90% of the particles relative to the weight (weight distribution), however, should lie within the intended size range.

A highly suitable measuring method is laser defraction for determination of particle size distribution. Commercial instruments permit measurement in air (Malvern Co. S3.01 Particle Sizer) or preferably in liquid media (LOT Co., Galai CIS 1). The prerequisite for measurement in liquids is that the polymer does not dissolve therein or the particles do not change in some other way during the measurement. An example of a suitable medium is a highly diluted (about 0.02%) aqueous Polysorbate 80 solution.

### Emulsifier

An emulsifier may be defined as a molecule or a substance comprising a balance of hydrophilic and hydrophobic (lipophilic) properties. This may also be called an amphiphilic property. Emulsifiers may be characterized by their HLB values (HLB stands for hydrophilic-lipophilic balance)

The HLB, introduced by Griffin in 1950, is a measure of the hydrophilicity of lipophilicity of nonionic surfactants. It may be determined experimentally by the phenol titration method of Marszall; cf. “Parfümerie, Kosmetik”, Volume 60, 1979, pp. 444-448; further literature references are in Römpp, Chemie-Lexikon, 8^{th} ed. 1983, p. 1750. See also, for example, U.S. Pat. No. 4,795,643 (Seth). An HLB (hydrophilic/lipophilic balance) can be determined exactly only for nonionic emulsifiers. For anionic emulsifiers, this value may be determined arithmetically but is virtually in most cases above or well above 20.

### Process

The invention is concerned with a process for preparing aqueous dispersions comprising, consisting essentially of or consisting at least of the components (a), (b) and ((c) or (d)) or both, ((c) and (d)),
(a) a copolymer consisting of free-radical-polymerized 01- to 04-esters of acrylic or methacrylic acid and alkyl(meth)acrylate monomers with tertiary ammonium groups in the alkyl side groups,
(b) 5 to 25% by weight, based on (a), of a C₁₂- to C₁₈-monocarboxylic acid or a C₁₂- to C₁₈-alcohol,
(c) 0 to 10% by weight based on (a) of a dicarboxylic acid having 3 to 10 carbon atoms,
(d) 0 to 20, preferably 0 to 15% by weight, based on (a), of an emulsifier having an HLB of at least 14
   by dispersing the components in water, preferably in pre-heated water or in cold water at room temperature (about 22° C.) and subsequent addition of preheated water,
   where the water either directly or after mixing has a temperature from 30 to 85, preferably more than 40 and up to 85, most preferably 40-60° C.,
   where the solid content of the dispersion is from 5 to 40, preferably 5 to 30, more preferably 10 to 20% by weight and
   where a clear colloidal dispersion is obtained in less than 30 min, preferably in less than 25 min, in less than 20 min or in less than 15 min.

### Component (a)

Component (a) is a copolymer consisting of free-radical-polymerized C1- to C4-esters of acrylic or methacrylic acid and alkyl(meth)acrylate monomers with tertiary amino groups in the alkyl side groups.

Preferably component (a) is a copolymer composed of polymerized units of 30 to 80% by weight of C₁- to C₄-alkyl esters of acrylic or of methacrylic acid and 70 to 20% by weight of alkyl(meth)acrylate monomers having a tertiary amino group in the alkyl radical.

Preferably component (a) is a copolymer composed of polymerized units of 20-30% by weight of methyl methacrylate, 20-30% by weight of butyl methacrylate and 60-40% by weight of dimethylaminoethyl methacrylate.

Preferably the component (a) is present in powder form with a mean particle size from 1-40, preferably from 5 to 25 μm.

Component (a) is an amino(meth)acrylate copolymer that may be composed partly or fully of alkyl acrylates and/or alkyl methacrylates having a tertiary amino group in the alkyl radical. Suitable (meth)acrylate copolymers are known, for example, from EP 0 058 765 B1.

Suitable monomers with functional tertiary amino groups are detailed in U.S. Pat. No. 4,705,695, column 3 line 64 to column 4 line 13. Mention should be made in particular of dimethylaminoethyl acrylate, 2-dimethylaminopropyl acrylate, dimethylaminopropyl methacrylate, dimethylaminobenzyl acrylate, dimethylaminobenzyl methacrylate, (3-dimethylamino-2,2-dimethyl)propyl acrylate, dimethylamino-2,2-dimethyl)propyl methacrylate, (3-diethylamino-2,2-dimethyl)propyl acrylate and diethylamino-2,2-dimethyl)propyl methacrylate. Particular preference is given to dimethylaminoethyl methacrylate.

A specifically suitable commercial amino (meth)acrylate copolymer is, for example, formed from 25% by weight of methyl methacrylate, 25% by weight of butyl methacrylate and 50% by weight of dimethylaminoethyl methacrylate (EUDRAGIT® E100 or EUDRAGIT® E PO (powder form, with an average particle size of around 15 μm). EUDRAGIT® E100 and EUDRAGIT® E PO are water-soluble below approx. pH 5.0 and are thus also gastric juice-soluble.

Suitable copolymers may be the “amino methacrylate copolymer (USP/NF)”, “basic butylated methacrylate copolymer (Ph. Eur)” or “aminoalkyl Methacrylate Copolymer E (JPE)” which are of the EUDRAGIT® E type.

A further suitable (meth)acrylate copolymer with tertiary amino groups may be, for example, formed from 50-60, preferably 55% by weight of methyl methacrylate and 40-50, preferably 45% by weight of diethylaminoethyl methacrylate (s. WO2009016258, WO2010139654 and WO2012041788A1).

### Component (b)

Component (b) is 5 to 25, preferably 10 to 20% by weight, based on component (a), of a C₁₂- to C₁₈-monocarboxylic acid or a C₁₂- to C₁₈-alcohol.

Component (b) may be lauric acid, palmitic acid, stearic acid, lauryl alcohol, palmityl alcohol or stearyl alcohol.

### Component (c)

Component (c) is a dicarboxylic acid having 3 to 10 carbon atoms.
Component (c) is an optional component and may be comprised or contained in amounts of 0 to 10, 1 to 10 preferably 0 to 5% by weight, based on component (a).
Component (c) may be for instance fumaric acid, tartaric acid, succinic acid or any mixtures thereof.

### Component (d)

Component (d) is an emulsifier having an HLB of at least 14.

Component (d) is an optional component and may be comprised or contained in amounts of 0 to 20, preferably 5 to 20% by weight, based on component (a).

Component (d) may be for instance sodium lauryl sulfate or polysorbate 80.

Preferred emulsifiers in respect to component (d) are non-ionic or anionic emulsifiers. Further preferred, the emulsifiers in respect to component (b) may be selected from but not limited to the group consisting of fatty alkyl sulfates, preferably sodium laurylsulfate, sodium cetylstearylsulfate, saccharose stearate, poloxamers, polysorbates, especially polysorbate 80 (Tween® 80) or mixtures thereof.

### Further Excipients

The dispersion may comprise or contain further excipients selected from the groups of antioxidants, brighteners, binding agents, flavouring agents, flow aids, fragrances, glidants, penetration-promoting agents, pigments, plasticizers, polymers, pore-forming agents or stabilizers. The further excipients are different from the components (a) to (d).

The dispersion may comprise or contain talc, Mg-stearate or silica or any combinations thereof as further excipients.

The dispersions may additionally comprise one or more excipients selected from 30 to 120% by weight, based on (a), talc, 10 to 100% by weight, based on (a), Mg-stearate or 10 to 100% by weight, based on (a), silica.

The further excipients may be added with the components (a), (b) and ((c) or (d)) or both, ((c) and (d)), to preheated water or to cold water (room temperature about 22° C.), to which hot water is added subsequently until the desired mixing temperature is reached, and may be homogenized separately from the other components in a part of the water and may then be added afterwards.

### Dispersing the Components in Water

Dispersing the components in water shall mean the more or less homogenous distribution of the solid or liquid components in a certain amount of water, preferably under stirring by means of stirrer or homogenizer equipment. The components (a) and (b) and optionally (c) or (d) or both and optionally the further excipient(s) may be dispersed all together or one after the other in one portion of water or where applicable also separately in different sub-portions of water which are subsequently added to each other.

### Water Temperature

The water used to disperse the components and optionally the further excipient(s) therein, until a clear colloidal dispersion is obtained, may have a temperature from 35 to 85, 35 to 60, 40 to 70, 40 to 60, preferably more than 40 and up to 70, more than 40 and up to 60, 42 to 60, 45 to 60, 48 to 60, 50 to 60° C. If the dispersion has a temperature of not more than 60° C., maybe from 30 to 60 or 45 to 60° C., it can be without cooling down advantageously directly used for spray applications. Preheated water may be used to disperse the components (a) and (b) and optionally (c) or (d) or both and optionally the further excipient(s). The components and optionally the further excipient(s) may as well be dispersed first in (cold) water, for instance at room temperature at about 22° C., to which hot water is added subsequently to obtain a mixing temperature of more than 40 and up to 70, more than 40 and up to 60, 42 to 60, 45 to 60, 48 to 60, 50 to 60° C.

### Mixing

The components are preferably mixed by means of stirrer or homogenizer equipment. The components are preferably mixed either under gentle stirring (with less than 100 rpm stirrer speed), or under vigorous stirring (100 to 500 rpm stirrer speed, for instance by an over head stirrer) or by using a high shear mixer respectively by applying high shear mixing. The high shear mixer may be used to apply stirring and shear forces.

### Preferred Dispersions

A preferred dispersion may comprise or contain
Component (a) with
component (a) with
(b) 5 to 25% by weight based on (a) stearic acid,
(d) 5 to 20% by weight based on (a) sodium lauryl sulfate.

Preferably the water used for dispersing the components (a), (b) and (d) may have a temperature of more than 40 and up to 85, more than 40 and up to 70, more than 40 and up to 60, 42 to 60, 45 to 60, 48 to 60 or 50 to 60° C.

The dispersion may additionally comprise one or more excipients selected from 30 to 120% by weight, based on (a), talc, 10 to 100% by weight, based on (a), Mg-stearate or 10 to 100% by weight, based on (a), silica (Syloid®). Talc, Mg-stearate and/or silica may be dispersed together with components (b) and (d) or separately in water within the same temperature ranges.

Another preferred dispersion may comprise or contain
component (a) with
(b) 5 to 25% by weight based on (a) stearic acid,
(c) 1 to 10% by weight based on (a) tartaric acid.

Preferably the water used for dispersing the components (a), (b) and (c) may have a temperature from 35 to 85, 40 to 70, from more than 40 to 85, more than 40 and up to 70, more than 40 and up to 60, 42 to 60, 45 to 60, 48 to 60 or 50 to 60° C. The dispersion may additionally comprise one or more excipients selected from 30 to 120% by weight, based on (a), talc, 10 to 100% by weight, based on (a), Mg-stearate or 10 to 100% by weight, based on (a), silica (Syloid®). Talc, Mg-stearate and/or silica may be dispersed together with components (b) and (d) or separately in water within the same temperature ranges. For this kind of dispersions the preparation time is advantageously reduced when the heated water is used in combination with high shear mixing as disclosed in here.

### Solid Content

The solid content of the aqueous dispersion may be in the range from 5 to 30, preferably in the range of 10 to 20% by weight. Water normally adds up to 100% by weight. Thus the water content may be in the range of 70-95, preferably in the range of 80 to 90% by weight.

### Advantages

The disclosed process is especially of advantage for dispersions comprising or containing substances with high melting points, such as stearic acid as a component (b). The melting point of stearic acid is in the range of 53-60° C. On the other hand component (a), an amino(meth)acrylate copolymer, such as EUDRAGIT® E or EUDRAGIT® E PO, has a glass transition temperature (Tg) of about 48° C. Without being bound to a theory the inventors assume that the combination of hot water, optionally combined with high shear mixing, promotes physical reactions among the ingredients or components that lead to shortened preparation times for the dispersions. The application of high-shear mixers is usually associated with large amounts of foam generation in preparations containing emulsifiers. Use of hot water in such cases is of special advantage as it avoids use of otherwise undesirable antifoaming agents or at least reducing foam formation to acceptable level.

Surprisingly it was also found that this tendency of reduction in process time is not directly proportional to the temperature of water, but was limited to a rather narrow temperature range. This was by no means to be foreseen and is of practical advantage. Especially for those above mentioned preferred mixtures or dispersions that are in practice widely used, comprising component (a), 5 to 20% by weight stearic acid and 5 to 15% by weight sodium lauryl sulfate or comprising component (a), 5 to 20% by weight stearic acid and 1 to 10% by weight tartaric acid, the process is of advantage.

### EXAMPLES

EUDRAGIT® E PO (EPO) is a copolymer composed of polymerized units of 25% by weight of methyl methacrylate, 25% by weight of butyl methacrylate and 50% by weight of dimethylaminoethyl methacrylate in powder form, with an average particle size of around 15 μm.

### Excipients

All excipients were used in pharmaceutical quality

### Example 1

### Tartaric Acid Formulation

| Ingredients | |
|---|---|
| EUDRAGIT ® E PO | Quantity (% on dry polymer) |
| Stearic acid | 15% |
| Tartaric acid | 3% |
| Talc | 50% |
| Water | Add up in total to 100% |

Solid Content 15% w/w

| Example 1/METHOD | PROCEDURE | OBSERVATIONS |
|---|---|---|
| Use of water at room | 1. In 70% of water tartaric acid was dissolved | In normal water stearic acid and EPO were not |
| temperature (ca. 22° C.) | 2. Stearic acid and EPO is then successively added under | disperse easily hence high shear is required and |
| | continuous homogenization for 30 min | time required is 30 min |
| | 3. Talc and pigments were separately homogenized in remaining | High foam generation which require more than 2 |
| | quantity of water for 30 minutes using Ultra-Turrax ®. | hours to settle |
| | 4. Step 3 dispersion is then added to step 2 dispersion | e.g -Initial volume of dispersion (with foam) = 680 ml |
| | | Final volume (after foam disappears) = 450 ml |
| | | Total volume of foam = 230 ml |
| | | Few small particles observed on the surface |
| | | when foam got disappeared |
| Use of hot water (55° C.) | 1. In 70% of Hot water (55° C.) tartaric acid was dissolved | In hot water dispersibility of stearic acid and EPO |
| | 2. Stearic acid and EPO is then successively added under | is improved and time required to prepare dispersion |
| | continuous homogenization for 15 min | is 15 min |
| | 3. Talc and pigments were separately homogenized in remaining | Foam formation was reduced which settled in |
| | quantity of water for 30 minutes using Ultra-Turrax ®. | 45 minutes |
| | 4. Step 3 dispersion is then added to step 2 dispersion | e.g -Initial volume of dispersion (with foam) = 600 ml |
| | | Final volume (after foam disappears) = 450 ml |
| | | Total volume of foam = 150 ml |
| | | Clear colloidal dispersion was obtained, when |
| | | foam got disappeared |

| | | | Time | | Time | | |
|---|---|---|---|---|---|---|---|
| | | | Taken to | | Taken | | |
| | | | form clear | Foam Volume | to | | |
| | | | colloidal | Initial volume − | settle | | |
| | | Temperature | dispersion | final volume | foam | | |
| Formulation | Stirrer | (degree C.) | (min) | (ml) | (min) | | Observations |
| Tartaric acid | High Shear | ca. 22 | 30 | 680 − 450 = 230 | >120 | min | Clear colloidal dispersion |
| formulation | Mixer | 40 | 20 | 670 − 450 = 170 | 80 | | Clear colloidal dispersion |
| | (Homogenizer) | 50 | 15 | 600 − 450 = 150 | 60 | | Clear colloidal dispersion |
| | | 55 | 15 | 610 − 450 = 160 | 60 | | Clear colloidal dispersion |
| | | 60 | 15 | 610 − 450 = 160 | 60 | | Clear colloidal dispersion |
| | | 70 | 15 | 610 − 450 = 160 | 60 | | Clear colloidal dispersion |
| | | 90 | — | — | — | | Lumps |

| | | | Time | | | Time | |
|---|---|---|---|---|---|---|---|
| | | | Taken to | | | Taken | |
| | | | form clear | | Foam Volume | to | |
| | | | colloidal | | Initial volume − | settle | |
| | | Temperature | dispersion | | final volume | foam | |
| Formulation | Stirrer | (degree C.) | (min) | | (ml) | (min) | Observations |
| Tartaric acid | Overhead | ca. 22 | >150 | min | 525 − 450 = 75 | 35 | Clear colloidal dispersion |
| formulation | Stirring | 50 | 120 | | 480 − 450 = 30 | 20 | Clear colloidal dispersion |
| | | 55 | 120 | | 480 − 450 = 30 | 20 | Clear colloidal dispersion |
| | | 60 | 120 | | 480 − 450 = 30 | 20 | Clear colloidal dispersion |
| | | 70 | 120 | | 480 − 450 = 30 | 20 | Clear colloidal dispersion |
| | | 90 | — | | — | — | Lumps |

### Example 2

### Standard EPO Formulation

| Ingredients | |
|---|---|
| EUDRAGIT EPO | Quantity (% on dry polymer) |
| Stearic acid | 15% |
| Sodium lauryl sulphate | 10% |
| Talc | 50% |
| Water | add up in total to 100% |

Solid Content 15% w/w

| METHOD | PROCEDURE | OBSERVATIONS |
|---|---|---|
| Use of water at room | 1. In 70% of water sodium lauryl sulphate was added, and then | Time required for the preparation of |
| temperature (ca. 22° C.) | stirred for 5 minutes using overhead stirrer at slow speed | dispersion (about 3 hours) |
| | 2. Stearic acid and EPO is then sucessively added under continuous | Require slow addition of ingredient |
| | stirring for about 3 hours. | Risk of lump formation |
| | 3. Talc and pigments were separately homogenized in remaining | Few small particles remains undispersed |
| | quantity of water for 30 minutes using an ULTRA_TURRAX ®. | |
| | 4. Step 3 dispersion is then added to step 2 dispersion | |
| | Solid content- 15% w/w | |
| Use of hot water (50° C.) | 1. In 70% of Hot water (50 degree C.) sodium lauryl sulphate was | Use of Hot Water (50° C.) gave colloidal |
| | added, and then homogenized for 5 minutes using overhead stirrer | dispersion by using overhead stirred at slow |
| | at slow speed | speed without foam formation in 15 min |
| | 2. Stearic acid and EPO is then sucessively added under continuous | |
| | stirring for 15 minutes. | |
| | 3. Talc and pigments were separately homogenized in remaining | |
| | quantity of water for 30 minutes using an ULTRA_TURRAX ®. | |
| | 4. Step 3 dispersion is then added to step 2 dispersion. | |
| | Solid content- 20% w/w | |

| | | | Time Taken | | Time | |
|---|---|---|---|---|---|---|
| | | | to form | Foam Volume | Taken | |
| | | | clear | Initial | to | |
| | | | colloidal | volume − | settle | |
| | | Temperature | dispersion | final volume | foam | |
| Formulation | Stirrer | (degree C.) | (min) | (ml) | (min) | Observations |
| Std EPO | High Shear | ca. 22 | 30 | 540 − 460 = 80 | 60 | Clear colloidal dispersion |
| Formulation | Mixer | 40 | 15 | 530 − 460 = 70 | 50 | Clear colloidal dispersion |
| | (homogenizer) | 50 | 15 | 520 − 460 = 60 | 45 | Clear colloidal dispersion |
| | | 55 | 15 | 520 − 460 = 60 | 45 | Clear colloidal dispersion |
| | | 70 | 10 | 520 − 460 = 60 | 40 | Clear colloidal dispersion |
| | | 90 | 5 | 520 − 460 = 60 | 40 | Initially milky white Clear |
| | | | | | | on standing |

| | | | Time Taken | | Time | |
|---|---|---|---|---|---|---|
| | | | to form | Foam Volume | Taken | |
| | | | clear | Initial | to | |
| | | | colloidal | volume − | settle | |
| | | Temperature | dispersion | final volume | foam | |
| Formulation | Stirrer | (degree C.) | (min) | (ml) | (min) | Observations |
| Std EPO | Over | ca. 22 | 180 | 470 − 460 = 10 | 10 | Clear colloidal dispersion |
| Formulation | head | 40 | 15 | 480 − 460 = 20 | 10 | Clear colloidal dispersion |
| | Stirrer | 50 | 15 | 480 − 460 = 20 | 10 | Clear colloidal dispersion |
| | | 55 | 15 | 480 − 460 = 20 | 10 | Clear colloidal dispersion |
| | | 70 | 10 | 480 − 460 = 20 | 10 | Clear colloidal dispersion |
| | | 90 | — | — | — | Lumps |

1: A process for preparing aqueous dispersions comprising the components (a), (b) and ((c) or (d)) or both:
(a) a copolymer comprising a free-radical-polymerized C1- to C4-ester of acrylic or methacrylic acid and an alkyl(meth)acrylate monomer having at least one tertiary amino group as an alkyl side group;
(b) 5 to 25% by weight, based on (a), of a C12- to C18-monocarboxylic acid or a C12- to C18-alcohol;
(c) 0 to 10% by weight based on (a) of a dicarboxylic acid having 3 to 10 carbon atoms; and
(d) 0 to 20% by weight, based on (a), of an emulsifier having an HLB of at least 14,
the process comprising dispersing the components in water to form the aqueous dispersion,
wherein:
the water has a temperature of more than 40 and up to 85° C.;
a solid content of the dispersion is from 5 to 40% by weight; and
a clear colloidal dispersion is obtained in less than 30 min.

2: The process of claim 1, wherein the component (a) is a copolymer comprising polymerized units of 30 to 80% by weight of the C₁- to C₄-alkyl ester of acrylic or of methacrylic acid and 70 to 20% by weight of then alkyl(meth)acrylate having a tertiary amino group in the alkyl radical.

3: The process of claim 1, wherein the component (a) is a copolymer comprising polymerized units of 20-30% by weight of methyl methacrylate, 20-30% by weight of butyl methacrylate and 60-40% by weight of dimethylaminoethyl methacrylate.

4: The process of claim 1, wherein the component (a) is present in powder form with an average particle size of 1-40 μm.

5: The process of claim 1, wherein the component (b) is lauric acid, palmitic acid, stearic acid, lauryl alcohol, palmityl alcohol or stearyl alcohol.

6: The process of claim 1, wherein the component (c) is fumaric acid, tartaric acid, succinic acid or mixtures thereof.

7: The process of claim 1, wherein the component (d) is sodium lauryl sulfate or polysorbate 80.

8: The process of claim 1, wherein the dispersion further comprises at least one excipient selected from the group consisting of an antioxidant, a brightener, a binding agent, a flavoring agent, a flow aid, a fragrance, a glidant, a penetration-promoting agent, a pigment, a plasticizer, a polymer, a pore-forming agent, a lubricant and a stabilizer.

9: The process of claim 1, wherein the dispersion further comprises talc.

10: The process of claim 1, wherein the components are dispersed into either preheated water or a mixture of water at room temperature and heated water.

11: The process of claim 1, wherein the components are dispersed into the water with a high shear mixer.

12: The process of claim 1, wherein the dispersed components include the component (a) with
(b) 5 to 25% by weight, based on (a), stearic acid,
(d) 5 to 20% by weight, based on (a), sodium lauryl sulfate.

13: The process of claim 1, wherein the dispersed components include the component (a) with
(b) 5 to 25% by weight, based on (a), stearic acid,
(c) 1 to 10% by weight, based on (a), tartaric acid.

14. The process of claim 12, wherein the components dispersions further comprise one or more excipients selected from the group consisting of 30 to 120% by weight, based on (a), talc, 10 to 100% by weight, based on (a), Mg-stearate and 10 to 100% by weight, based on (a), silica.
